(19) United States
(12) Patent Application Publication
(10) Pub. No.: US 2026/0123988 A1
(43) Pub. Date: May 7, 2026

(54) HAND-ACTIVATED MICROWAVE PROBE HANDLES

(71) Applicant: Neuwave Medical, Inc., Madison, WI (US)

(72) Inventors: Jason BRUNKOW, Mcfarland, WI (US); Mark THOM, Verona, WI (US); Christopher UNSETH, Sun Prairie, WI (US)

(73) Assignee: Neuwave Medical, Inc., Madison, WI (US)

(21) Appl. No.: 18/935,198

(22) Filed: Nov. 1, 2024

(51) Int. Cl. A61B 18/18; A61B 18/00; A61B 90/50

(52) U.S. Cl. CPC A61B 18/1815; A61B 90/50; A61B 2018/00023; A61B 2018/00077; A61B 2018/00178; A61B 2018/00577; A61B 2018/00708; A61B 2018/00922; A61B 2018/1861

(57) ABSTRACT

A system is disclosed including an ablation probe that includes an antenna, a controller in communication with the ablation probe, and a holster device. The holster device is configured to removably receive the ablation probe, wherein the holster device further comprises a switch and, when the ablation probe is mounted in the holster device, the controller is operable to activate the ablation probe upon activation of the switch.

### BACKGROUND

Ablation is an important therapeutic strategy for treating certain tissues, such as benign and malignant tumors, cardiac arrhythmias, cardiac dysrhythmias, and tachycardia. Some ablation systems utilize radio frequency (RF) energy as the ablating energy source. However, RF energy has several limitations, including the rapid dissipation of energy in surface tissues resulting in shallow “burns” and failure to access deeper tumor or arrhythmic tissues. Another limitation of RF ablation systems is the tendency of eschar and clot formation on the energy emitting electrodes, which limits further deposition of electrical energy.

More recently, microwave energy is being used as the ablating energy source in ablation systems. Microwave energy is an effective energy source for heating biological tissues, and is used in applications such as cancer treatment and preheating of blood prior to infusions. One advantage of microwave energy over RF is the deeper penetration into tissue, insensitivity to charring, lack of necessity for grounding, more reliable energy deposition, faster tissue heating, and the capability to produce much larger thermal lesions than RF, which greatly simplifies the actual ablation procedures.

When performing an ablation procedure, probes are utilized to deliver the microwave energy, and such probes are sometimes activated by foot pedals or by computer touch screens or peripheral devices. In some applications, for example, the physician may activate the ablation probe via a foot petal during a procedure, or the physician may ask an assistant (e.g., a nurse) to activate the ablation probe via a computer touch screen during the procedure. Moreover, in some procedures, a pair of ablation probes positioned side by side are utilized and, in these use cases, the pair of ablation probes are activated independently via the foregoing foot pedal or computer means.

It could be helpful, however, to allow for activation of one or more ablation probes by hand during an ablation treatment, which would provide the physician a great degree of precision when using the ablation probe(s), while at the same time still allowing for traditional means of activating the ablation probe(s) (e.g., via foot pedal, computer touch screen, etc.) which may still be beneficial in some use cases. Accordingly, there is a need for improved systems and methods that enable a physician to activate one or more ablation probes via hand.

### SUMMARY OF THE DISCLOSURE

Various details of the present disclosure are hereinafter summarized to provide a basic understanding. This summary is not an exhaustive overview of the disclosure and is neither intended to identify certain elements of the disclosure, nor to delineate the scope thereof. Rather, the primary purpose of this summary is to present some concepts of the disclosure in a simplified form prior to the more detailed description that is presented hereinafter.

According to an embodiment consistent with the present disclosure, a system includes an ablation probe that includes an antenna, a controller in communication with the ablation probe, and a holster device configured to removably receive the ablation probe, the holster device including a switch. When the ablation probe is mounted to the holster device, the switch is placed in communication with the controller such that actuating the switch correspondingly activates the ablation probe.

In another embodiment, a method includes providing an ablation probe that includes a handle housing, an antenna extending distally from the handle housing, and a first communication port mounted to the handle housing and including one or more first electrical contacts in communication with a controller. The method further includes mounting the ablation probe to a holster device that includes a docking bay sized to receive the handle housing, a switch, and a second communication port arranged within the docking bay and including one or more second electrical contacts in communication with the switch. The method further includes aligning and mating the one or more first electrical contacts with the one or more second electrical contacts and thereby placing the switch in communication with the controller, and actuating the switch and thereby activating the ablation probe.

In a further embodiment, a system includes an ablation probe that includes an antenna and a first electrical contact, a controller in communication with the first electrical contact, and a holster device having a switch and a second electrical contact in communication with the switch. The holster is configured to removably receive the ablation probe and the second electrical contact is arranged to contact the first electrical contact when the ablation probe is mounted to the holster, and wherein the controller is operable to activate the ablation probe upon activation of the switch.

Any combinations of the various embodiments and implementations disclosed herein can be used in a further embodiment, consistent with the disclosure. These and other aspects and features can be appreciated from the following description of certain embodiments presented herein in accordance with the disclosure and the accompanying drawings and claims.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, without departing from the scope of this disclosure.

FIG. 1 is a block diagram of an energy delivery system that may be used in accordance with the principles of the present disclosure.

FIG. 2 is a schematic diagram of an example ablation probe that may be used in accordance with the principles of the present disclosure.

FIG. 3 depicts an ablation probe, according to one or more embodiments of the present disclosure.

FIG. 4 depicts a holster device for use with an ablation probe, according to one or more embodiments of the present disclosure.

FIG. 5 depicts an alternate holster device for use with a pair of ablation probes, according to one or more embodiments of the present disclosure.

### DETAILED DESCRIPTION

The present disclosure is related to systems and methods for delivering energy to tissue for ablation operation and, more particularly, to systems and methods for holding and activating ablation probes.

The present disclosure is related to comprehensive systems, devices, and methods for delivering energy (e.g., microwave energy, radiofrequency energy, laser, focused ultrasound, plasma, etc.) to tissue for a wide variety of applications including medical procedures (e.g., percutaneous or surgical). Example medical procedures that may benefit from the embodiments described herein include, but are not limited to, tissue ablation, resection, cautery, vascular thrombosis, intraluminal ablation of a hollow viscus, cardiac ablation for treatment of arrhythmias, electrosurgery, tissue harvest, cosmetic surgery, intraocular use, or any combination thereof.

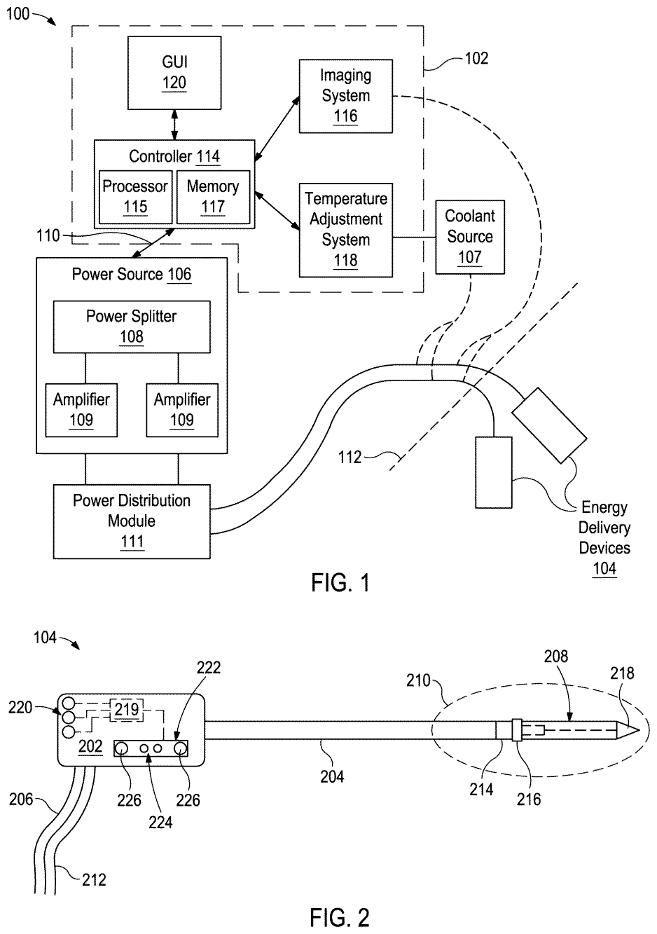

FIG. 1 is a block diagram of an example energy delivery system 100 that may incorporate the principles of the present disclosure. As illustrated, the energy delivery system 100 (hereafter “the system 100”) includes a control system 102 and one or more energy delivery devices or “ablation probes” 104 (two shown) designed to deliver (emit) energy to a target tissue region of a patient. While two ablation probes 104 are shown, the system 100 may include only one ablation probe 104 or more than two ablation probes 104 (e.g. three, four, or five ablation probes). An example ablation probe 104 that can be used with the system 100 is described in U.S. Patent Application Publication No. 2021/0282851, entitled “ENERGY DELIVERY SYSTEMS AND USES THEREOF”, which published on Sep. 16, 2021, the disclosure of which is hereby incorporated by reference in its entirety.

The system 100 further includes a power source or generator 106 communicably coupled to the control system 102 and the ablation probes 104 to direct, control, and deliver (provide) electrical power thereto. In some applications, the power source 106 may include a power splitter 108 that receives power from an external power source (e.g. a wall outlet) and directs power to one or more amplifiers 109 (two shown), which amplify the voltage, current, or power from the power splitter 108 to an associated one of the ablation probes 104. While two amplifiers 109 are shown, each associated with a corresponding one of the ablation probes 104, the power source 106 may include less than two amplifiers (e.g. one amplifier) or more than two amplifiers (e.g. three, four, or five amplifiers, for example). Each amplifier 109 may be coupled to a corresponding ablation probe 104 via a power distribution module 111, which provides strain relief to the cabling extending from the amplifiers 109 to the ablation probes 104. The power distribution module 111 may be coupled to a structure in the operating room, such as a surgical bed, and may house connection hardware of the ablation probes 104.

The components of the system 100 are connected via one or more cables or transmission lines 110. Moreover, the ablation probes 104 are designed to operate within a sterile field facilitated by the use of a sterile field barrier 112 that separates the ablation probes 104 from the remaining components of the system 100. The sterile field barrier 112 creates the sterile field, which includes any region permitting access only to sterilized items (e.g., sterilized devices, sterilized accessory agents, sterilized body parts, etc.). The sterile field barrier 112 hinders entry of non-sterile items into the sterile field, and the ablation probes 104 are configured for operation within the sterile field.

FIG. 2 is a schematic diagram of an example ablation probe 104 that may be used in accordance with the principles of the present disclosure. As indicated above, the ablation probe 104 may be configured to deliver (emit) energy (e.g., microwave energy, radiofrequency energy, radiation energy) to a target tissue region. As illustrated, the ablation probe 104 includes a handle housing 202 and an elongate shaft or probe cannula 204 extending distally from the handle housing 202 (hereinafter, “the handle 202”).

A cable or cable assembly 206 may be operatively coupled to the handle 202 and configured to convey electrical power thereto. The cable assembly 206 may extend from the power distribution module 111 (FIG. 1), for example, and may provide the power sufficient to operate the ablation probe 104. An antenna 208 is provided at the distal end of the probe cannula 204 and receives electrical power from the cable assembly 206 to emit energy (e.g., microwave energy) to a target tissue region and thereby generate an ablation zone 210 (shown in dashed lines). The material of the antenna 208 is durable and provides a high dielectric constant. In some applications, the material of the antenna 208 is zirconium and/or a functional equivalent of zirconium. In at least one application, the ablation probe 104 includes two or more separate antennae 208 attached to the same or different power supplies. Accordingly, microwave energy may be emitted from the antenna 208 upon activation of the ablation probe 104.

In some applications, a cooling tube 212 is operatively coupled and configured to convey a cooling fluid or “coolant” to the handle 202. The handle 202 may be configured to control conveyance of the cooling fluid into and out of the probe cannula 204 to help regulate a temperature of the antenna 208 and the ablation zone 210. Example cooling fluids include, but are not limited to, water, glycol, air, inert gases (e.g., helium), carbon dioxide, nitrogen, sulfur hexafluoride, ionic solutions (e.g., sodium chloride with or without potassium and other ions), dextrose in water, Ringer's lactate, organic chemical solutions (e.g., ethylene glycol, diethylene glycol, or propylene glycol), oils (e.g., mineral oils, silicone oils, fluorocarbon oils), liquid metals, freons, halomethanes, liquified propane, other haloalkanes, anhydrous ammonia, sulfur dioxide, or any combination thereof.

In some applications, the ablation probe 104 may include a stick region 214, alternately referred to as a “tissue-loc” region, provided on the probe cannula 204 at or near the antenna 208. The stick region 214 is designed to attain and maintain a temperature that accommodates adherence of a tissue region onto its surface. More specifically, the stick region 214 may operate as an anchoring element having a circulating agent or “coolant” (e.g., a gas delivered at or near its critical point; CO₂) that freezes the interface between the stick region 214 and the adjacent tissue, thereby sticking (maintaining, locking, etc.) the antenna 208 in place during operation, which in turn helps to maintain position of the ablation zone 210 within the patient. The coolant may be provided to the stick region 214 via the cooling tube 212 from a coolant source 107, for example. Once a pre-determined low temperature is reached at the stick region 214, contact with adjacent tissue causes the tissue to adhere to the stick region 214, thereby resulting in attachment of the ablation probe 104 to the tissue. During ablation, as the tissue warms, the antenna 208 remains secured to the tissue region due to tissue desiccation and charring. The stick region 214 may be made of any material able to attain and maintain a temperature such that upon contact with tissue induces adherence of the tissue onto the stick region 214. Example materials for the stick region 214 include, but are not limited to, a metal.

In some applications, the ablation probe 104 may further include a plug region 216 provided on the probe cannula 204 at or near the antenna 208. In at least one application, as depicted, the plug region 216 may be provided distal to the stick region 214 and otherwise interposing the stick region 214 and the antenna 208. The plug region 216 may be configured to prevent a reduction in temperature resulting from the cooled probe cannula 204 and the stick region 214 from affecting (e.g., reducing) the temperature within the antenna 208. Accordingly, the plug region 216 separates interior portions of the ablation probe 104 to prevent cooling or heating of a portion or portions of the ablation probe 104 while permitting cooling or heating of other portions. The plug region 216 may be made of an insulative material capable of being in contact with a material or region having a low temperature without having its temperature significantly reduced. Example insulative materials for the plug region 216 include, but are not limited to, a synthetic polymer (e.g., polystyrene, polyicynene, polyurethane, polyisocyanurate), aerogel, fiberglass, cork, or any combination thereof.

In some applications, the ablation probe 104 may further include a sharp stylet tip or “stylet” 218 positioned at the distal end of the antenna 208 and otherwise forming the distal end of the ablation probe 104. When included, the stylet 218 is designed to facilitate percutaneous insertion of the ablation probe 104. The stylet 218 may be made of a variety of rigid or hardened materials including, but not limited to, a hardened resin, a metal (e.g., titanium or an equivalent of titanium, stainless steel, etc.), a ceramic, or any combination thereof. In at least one application, the stylet 218 may be brazed to zirconia or an equivalent of zirconia. In such applications, the stylet 218 may comprise an extension of a metal portion of the antenna 208 and may be electrically active.

In some applications, the ablation probe 104 may have a coaxial transmission line positioned within the antenna 208, and a coaxial transmission line connecting with the antenna 208. In other embodiments, the ablation probe 104 may comprise a triaxial microwave probe with optimized tuning capabilities. The ablation probe 104 may be the same as or similar to any of the energy delivery devices described in U.S. Pat. No. 11,638,607, entitled “ENERGY DELIVERY SYSTEMS AND USES THEREOF”, which issued on May 2, 2023, the contents of which are hereby incorporated by reference in their entirety herein.

Referring again to FIG. 1, the control system 102 is configured to monitor, control, and provide feedback concerning operation of the system 100. As illustrated, the control system 102 includes at least a controller 114, an imaging system 116, and a temperature adjustment system 118. The control system 102 may further include a graphical user interface (GUI) or display 120, such as a touchscreen interface, which can be accessed by a user (e.g., a surgeon, a nurse, bedside assist, etc.) to operate the system 100. In some applications, the control system 102 may be mounted to or otherwise form part of a portable cart or “procedure cart,” and the GUI 120 may be arranged in a display region for operating and/or monitoring the components of the system 100.

The controller 114 may include a processor 115 and a memory or memory device 117 comprising any storage media readable by the processor 115. The memory 117 may store software or software instructions executable by the processor 115 to carry out functions and operations of the system 100. Examples of the memory 117 include, but are not limited to, random access memory (RAM), read-only memory (ROM), computer chips, optical discs (e.g., compact discs (CDs), digital video discs (DVDs), etc.), magnetic disks (e.g., hard disk drives (HDDs), floppy disks, ZIP® disks, etc.), magnetic tape, and solid state storage devices (e.g., memory cards, “flash” media, etc.). As used herein, the term “computer readable medium” refers to any device or system for storing and providing information (e.g., data and instructions) to the processor 115. Examples of computer readable media include, but are not limited to, optical discs, magnetic disks, magnetic tape, solid-state media, and servers for streaming media over networks.

Based on instructions provided by the software, the controller 114 may be configured to regulate the amount of energy (e.g., microwave energy) provided to a tissue region by the ablation probes 104 by monitoring characteristics of the tissue region, such as the size and shape of a target tissue, the temperature of the tissue region, etc. The controller 114 interacts with the ablation probes 104 to raise or lower (e.g., tune) the amount of energy delivered to the tissue region. The controller 114 may also be configured to prime coolants for distribution into the ablation probes 104 such that the coolant is delivered at a desired temperature.

In some applications, the type of tissue being treated is inputted into the software for purposes of allowing the controller 114 to regulate (e.g., tune) the delivery of microwave energy to the tissue region based upon pre-calibrated methods for that particular type of tissue or tissue region. In other embodiments, however, the type of probe selected for the particular procedure may be specifically tuned to a specific tissue type, and projected (expected) ablation sizes may be based on tissue type. In such embodiments, the controller 114 may not control power delivery based on tissue type. In yet other embodiments, the controller 114 generates a chart or diagram based upon a particular type of tissue or tissue region displaying characteristics useful to a user of the system.

The controller 114 may allow a user to choose power, duration of treatment, different treatment algorithms for different tissue types, simultaneous application of power to multiple probes 104, coherent and incoherent phasing, etc. The controller 114 may also be configured to create a database of information (e.g., required energy levels, duration of treatment for a tissue region based on particular patient characteristics, etc.) pertaining to ablation treatments for a particular tissue region based upon previous treatments with similar or dissimilar patient characteristics.

The control system 102 further includes the imaging system 116, which is in communication with the controller 114 and comprises one or more imaging devices. Example imaging devices include, but are not limited to, endoscopic devices, stereotactic computer assisted neurosurgical navigation devices, thermal sensor positioning systems, motion rate sensors, steering wire systems, intraprocedural ultrasound, interstitial ultrasound, microwave imaging, acoustic tomography, dual energy imaging, fluoroscopy, computerized tomography magnetic resonance imaging, nuclear medicine imaging devices triangulation imaging, thermoacoustic imaging, infrared and/or laser imaging, or electromagnetic imaging. In some embodiments, the system 100 uses endoscopic cameras, imaging components, and/or navigation systems that permit or assist in placement, positioning, and/or monitoring of the ablation probes 104.

In some applications, the system 100 provides software configured for use with imaging equipment of the imaging system 116, such as CT, MRI, and ultrasound, and generate two-dimensional (2D) and or three-dimensional (3D) images viewable by a user on the GUI 120. In some embodiments, the imaging equipment software allows a user to make predictions based upon known thermodynamic and electrical properties of tissue, vasculature, and location of the antenna(s) 208 (FIG. 2). In some embodiments, the imaging software allows the generation of a 2D or 3D map of the location of a tissue region (e.g., tumor, arrhythmia), location of the antenna(s) 208, and to generate a predicted map of the ablation zone 210 (FIG. 2).

In some applications, the imaging system 116 may be configured to monitor ablation procedures, such as monitoring the amount of ablation occurring within a particular tissue region(s) undergoing a thermal ablation procedure. The monitoring includes, but is not limited to, MRI imaging, CT imaging, ultrasound imaging, nuclear medicine imaging, and fluoroscopy imaging. The software may be designed to automatically obtain images of a tissue region (e.g., MRI imaging, CT imaging, ultrasound imaging, nuclear medicine imaging, fluoroscopy imaging), automatically detect any changes in the tissue region (e.g., blood perfusion, temperature, amount of necrotic tissue, etc.), and based on the detection to automatically adjust the amount of energy delivered to the tissue region through the ablation probes 104.

The power source 106 may be configured to supply the energy required to operate the system 100. The power source 106 is also configured to supply energy to the ablation probes 104, such as microwave energy, radiofrequency energy, radiation, cryo energy, electroporation, high intensity focused ultrasound, or any combination thereof. The power source 106 supplies microwave energy to the ablation probes 104 for purposes of tissue ablation. More specifically, power may be supplied to the ablation probes 104, but the microwave energy is generated in a microwave generator and sent to the antenna 208. In some applications, the power source 106 may include one or more energy generators configured to provide as much as 140-150 watts of microwave power of a frequency of from 915 MHz to 5.8 GHz, although the present disclosure is not so limited. The power splitter 108 may comprise a power distribution system operable to distribute the energy from the power source 106 to the ablation probes 104. The power splitter 108 may be configured to provide varying energy levels to different regions of the ablation probes 104.

The temperature adjustment system 118 may be configured to use coolant systems (like the coolant source 107) and cooling fluids to help reduce undesired heating within and along the ablation probes 104. In particular, the temperature adjustment system 118 may include the coolant source 107 and the cooling tube 212 (FIG. 2) and may communicate with the handle 202 (FIG. 2) of each ablation probe 104 to control conveyance of the cooling fluid into and out of the probe cannula 204 (FIG. 2), and thereby help regulate a temperature of the antenna 208 (FIG. 2) and the ablation zone 210 (FIG. 2).

In some applications, the temperature adjustment system 118 may also be configured to communicate with the handle 202 to operate the stick region 214 and thereby attain and maintain a temperature that accommodates adherence of tissue onto its surface. For instance, in some embodiments, a user provides an input to an input interface, such as the GUI 120. Based on the user input, the temperature adjustment system 118 can control the coolant source 107 to provide coolant to the stick region 214, thereby adhering the stick region 214 to the adjacent tissue. Accordingly, activation of the ablation probe 104 may include circulating cooling fluids through the ablation probe 104 and, more specifically, causing cooling fluids to flow from the coolant source to the ablation probe 104, where such cooling fluid flows through the cannula 204 and to the stick region 214 of the ablation probe 104, after which the cooling fluid is circulated back to the coolant source 107.

The temperature adjustment system 118 may also be configured to continuously or intermittently monitor the real-time temperature of the ablation probes 104. In such embodiments, the temperature adjustment system 118 may communicate with one or more temperature sensors (e.g., thermocouples) terminating at various points along the probe cannula 204 (FIG. 2) and/or the antenna 208 (FIG. 2) of the ablation probe 104. Consequently, localized temperature may be monitored at several points along the antenna 208 to estimate ablation status, cooling status, or safety checks. In some applications, monitoring the temperature at several points along the antenna 208 may help determine the geographical characteristics of the ablation zone 210 (FIG. 2), such as diameter, depth, length, density, width, etc., based upon the tissue type, and the amount of power used in the ablation probe 104. In other embodiments, or in addition thereto, the temperature may be measured not only at specific points along the probe cannula 204, but continuously along its entire length.

In some embodiments, the probe cannula 204 includes a plurality of temperature sensors. A first temperature sensor can be placed at, or slightly proximal to, the antenna 208 to provide real-temperature measurements of the tissue being heated by the antenna 208. A second temperature sensor can be placed at, or adjacent to, the stick region 214 to provide real-time temperature measurements of the tissue that is being cooled, and thus adhered to, the stick region 214. A third temperature sensor can be proximal to the first and second temperature sensors along the cannula 204, such as at the point of entry into the skin, to provide real-time measurements of the patent's skin. The control system 102 can receive the temperature measurements from the temperature sensors to control the coolant systems and cooling fluids from the temperature adjustment system 118 to the stick region 214 and/or other cooling systems of the ablation probe 104.

The temperature adjustment system 118 may also be configured to monitor the temperature of a tissue region (e.g., tissue being treated, surrounding tissue). This may prove advantageous in helping to determine the status of the procedure (e.g., the end of the procedure). The temperature adjustment system 118 may communicate with the controller 114 to provide real-time temperature information to a user and display such measurements on the GUI 120. In at least one embodiment, based on the temperature data obtained by the temperature adjustment system 118, the controller 114 may be configured to autonomously adjust operation of the system 100 appropriately.

Microwave Ablation Prove Multi-Function Port with Accessory Dual-Probe Activation Handle

Referring again to FIG. 2, with continued reference to FIG. 1, the ablation probe 104 includes input/output circuitry 219 that may be housed within the handle 202, and the input/output circuitry 219 may be in communication with various components of the system 100, such as the power source 106, the coolant source 107, the temperature adjustment system 118, and/or the controller 114 detailed above. As shown in FIG. 2, the ablation probe 104 may further include one or more lights 220 mounted to the handle 202 and operatively coupled to (in communication with) the input/output circuitry 219. The lights 220 may be light emitting diodes (LEDs), for example, and the input/output circuitry 219 may be programmed to selectively illuminate one or more of the lights 220 to indicate specific operation of the ablation probe 220.

For example, one of the lights 220 may be illuminated when power is being supplied to the ablation probe 104, another one of the lights 220 may be illuminated when the antenna 208 is emitting energy, and another one of the lights 220 may be illuminated when coolant is circulating through the cannula 204 to anchor the antenna 208 to the adjacent tissue at the stick region 214, and thereby maintain position of the ablation zone 210, as generally detailed above. However, the input/output circuitry 219 may be differently programmed/configured to provide other indications, without departing from the present disclosure.

Also shown in FIG. 2, the ablation probe 104 may further include a communication port 222 mounted to the handle 202 and operably connected to (in communication with) the input/output circuitry 219. As mentioned above, the input/output circuitry 219 is operably connected to upstream components of the system 100, such as the controller 114, and, therefore, the communication port 222 is also in communication with such upstream components of the system 100. The input/output circuitry 219 is programmed to allow for the communication port 222 to control operation of the ablation probe 104. For example, the communication port 222 may be used to control whether power to the ablation probe 104 is “off” or “on”, to control whether coolant flow is “off” or “on”, or any other feature of the ablation probe 104.

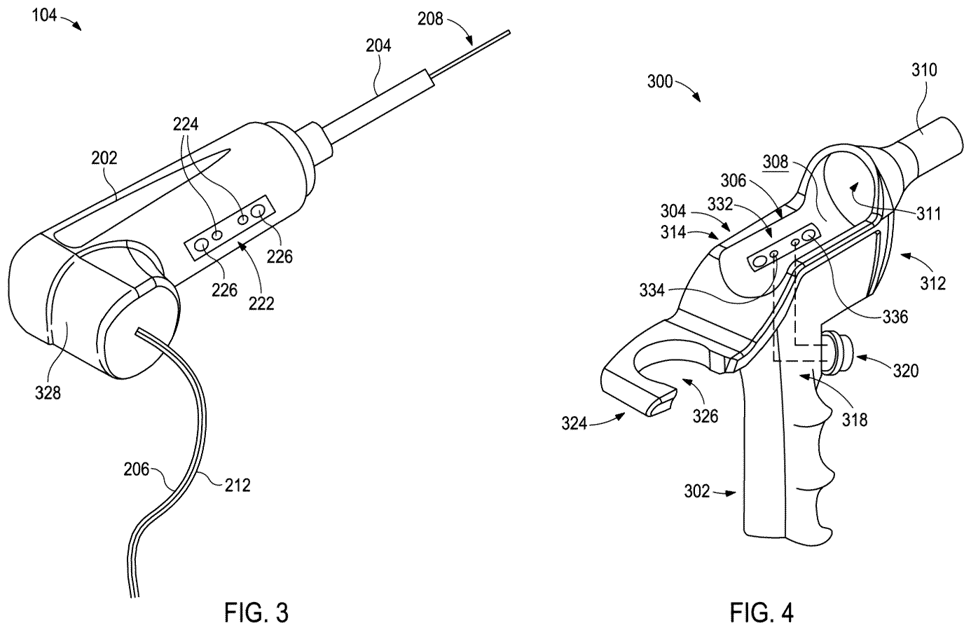

As shown in FIGS. 2 and 3, the communication port 222 includes electrical contacts or traces 224 that are in communication with the input/output circuitry 219. In the illustrated embodiment, the electrical contacts 224 include two electrical contacts/traces, with a first of the electrical contacts 224 being connected to an LED printed circuit board contained within the ablation probe 104, and with a second of the electrical contacts 224 being connected to ground. In embodiments, one or more additional electrical contacts 224 may be included, even if unused, to facilitate integration of additional functionality in the future.

Here, the controller 114 of the system 100 is configured to continually monitor the circuitry within the ablation probe 104 and, when the controller 114 determines that the circuitry within the ablation probe 104 is in a “high state” (or deactivated mode), the controller 114 will cause the ablation probe 104 to be (or remain) in a deactivated mode or idle state (i.e., where the ablation probe 104 is not ablating). In contrast, when the controller 114 determines that the circuitry within the ablation probe 104 is in a “low state” (or activated mode), the controller 114 will cause activation of the ablation probe 104. In an example, the “high state” (or deactivated mode) occurs when the controller 114 determines that the circuitry is subjected to (or experiencing) 3.3 Volts and the “low state” (or activated mode) occurs when the controller 114 ascertains that the circuitry is grounded (i.e., exposed to 0 Volts), and the ablation probe 104 is operable to ablate when the circuitry is grounded but, when the controller 114 determines that the Voltage has returned to “high state” of 3.3 Volts, the controller 114 causes the ablation probe 104 to stop ablating. Thus, as further described below, the circuitry within the ablation probe 104 may be in the “low state” (or the activated mode) when it is open (i.e., not complete), such that it is just grounded and the first of the electrical contacts 224 is disconnected.

In embodiments, the electrical contacts 224 may include one or more additional contacts. For example, the electrical contacts 224 may include a third contact/trace. Such additional, or third, contact/trace could be integrated with additional circuitry for controlling additional features or functions of the system 100. For example, inclusion of a third contact/trace could be utilizable for powering one or more of the light 220 arranged on the handle 202, for example, to provide indication of when the ablation probe 104 is ablating or performing one or more other functions, and/or inclusion of a third contact/trace could be utilizable controlling flow of the coolant to the stick region 214 of the ablation probe 104, etc. In an example where the third contract/trace is integrated into controlling functionality of the stick region, the controller 114 could recognize the “low state” and the “high state” to turn on coolant flow and turn off coolant flow, respectively, as described above with respect to activation and deactivation of the ablating feature of the ablation probe 104.

In some embodiments, the communication port 222 may also include one or more magnets 226 (two shown in FIGS. 2 and 3). In the illustrated embodiment, the electrical contacts 224 are arranged in a single file row and the magnets 226 are arranged on opposite ends of the row of electrical contacts 224. However, the electrical contacts 224 and/or the magnets 226 may be differently arranged. The magnets 226 may be utilized to help seat the ablation probe 104 within a button activated probe clip (sometimes referred to as a “holster device”) and maintain contact between the electrical contacts 224 of the ablation probe 104 and associated electrical contacts/traces contained within the holster device, as detailed below.

FIG. 4 depicts an example holster device 300 that can be used with the ablation probe 104, according to one or more embodiments. The holster device 300 may be designed and otherwise operable to removably receive the ablation probe 104. While FIGS. 1-2 schematically depict one embodiment of the ablation probe 104, FIG. 3 depicts another embodiment of the ablation probe 104 specifically configured to integrate with the holster device 300. Accordingly, the ablation probe 104 may be differently configured (designed) to be received by the holster device 300, as described in more detail below.

In the illustrated embodiment, the holster device 300 includes a handle portion 302 and a cradle portion 304 connected to the handle portion 302. In embodiments, the handle portion 302 and the cradle portion 304 are integrally formed as a monolithic structure. In other embodiments, however, the handle and cradle portions 302, 304 may comprise separate component parts that may be attached together. In at least one embodiment, the cradle portion 304 is removably attached to the handle portion 302.

As shown, the cradle portion 304 is configured to receive the handle 202 of the ablation probe 104. More specifically, the cradle portion 304 includes or otherwise defines a docking bay 306 within which the handle 202 of the ablation probe 104 may be received and mounted. The docking bay 306 is a recess in the cradle portion 304 that is defined by a mounting surface 308, which may be contoured and dimensioned to correspond to the geometry of the handle 202.

In the illustrated embodiment, the holster device 300 includes a cannula 310 through which the probe cannula 204 and the antenna 208 of the ablation probe 104 are able to extend when the ablation probe 104 is connected to the holster device 300. More specifically, the cannula 310 is arranged on the cradle portion 304 and structurally communicates with the docking bay 306. As illustrated, the cradle portion 304 includes a distal end 312 and a proximal end 314, and the cannula 310 extends distally from the distal end 312.

As shown, the cannula 310 is hollow and defines an interior bore or lumen 311 through which the probe cannula 204 and the antenna 208 may extend when the ablation probe 104 is mounted to the cradle portion 304. In embodiments, the probe cannula 204 of the ablation probe 104 contacts an inner bore surface of the lumen 311 when the ablation probe 104 is installed in the holster device 300.

In embodiments, the holster device 300 is configured to retain a cable of the ablation probe 104, such as either or both of the cable assembly 206 and/or the cooling tube 212. More specifically, the handle portion 302 of the ablation probe 104 may be configured to retain either or both of the cable assembly 206 and/or the cooling tube 212. For example, as discussed in more detail below, one or more slots (not shown) may be formed in the handle portion 302 and either or both of the cable assembly 206 and/or the cooling tube 212 may be received within (e.g., snap fit) the slot(s).

The holster device 300 also includes a switch 320 that is operable to activate the ablation probe 104 when the ablation probe 104 is properly seated/mounted in the holster device 300. In embodiments, the switch 320 includes a spring loaded trigger that is movable between a first or “activated” position and a second or “inactivated” position, and the spring loaded trigger may be biased to the inactivated position. When the switch 320 is transitioned to the activated position, such as by manually pressing or engaging the switch 320, the ablation probe 104 is activated. The antenna 208 outputs microwave energy upon activation of the ablation probe 104 via the switch 320. In embodiments, cooling fluid delivered to the ablation probe 104 via the cooling tube 212 may also be circulated through the ablation probe 104 (and, in particular, the antenna 208) upon activation of the ablation probe 104 via the switch 320. Manually releasing or disengaging from the switch 320 will allow the switch 320 to transition back to the inactivated position, in which the ablation probe 104 is deactivated.

In the illustrated embodiment, the switch 320 is arranged on the handle portion 302 of the ablation probe 104, for example, near an upper end 318 of the handle portion 302 proximate to the cradle portion 304. More specifically, the switch 320 may be arranged at the upper end 318 of the handle portion 302 at the junction where the handle portion 302 meets/joins the cradle portion 304.

The holster device 300 may also configured to retain the ablation probe 104 in order to inhibit accidental removal of the ablation probe 104 from the holster device 300. In some embodiments, for example, the holster device 300 may include a retainer 324 extending proximally from the proximal end 314 of the cradle portion 304. As illustrated, the retainer 324 includes or defines an opening 326 within which a proximal portion 328 of the ablation probe 104 may be received and retained (e.g., via a snap fit engagement). When the proximal portion 328 is disposed within the opening 326, the retainer 324 at least partially surrounds the proximal portion 328 and thereby inhibits removal of the ablation probe 104 from the holster device 300 until sufficient force is applied to the ablation probe 104 to overcome the snap fit configuration of the retainer 324 against the proximal portion 328. Also, when the proximal portion 328 of the ablation probe 104 is inserted into the opening 326 of the retainer 324, the proximal portion 328 extends downward in a direction substantially similar (or parallel) to the handle portion 302 to thereby similarly direct the cable assembly 206 and/or the cooling tube 212 downward in a direction substantially similar (or parallel) to the handle portion 302, which in turn facilitates cable management.

The holster device 300 may further include a communication port 332 that is mounted to the holster device 300 and operably connected to (in communication with) the switch 320. As illustrated, the communication port 332 is arranged within the docking bay 306 of the cradle portion 304. The holster device 300 may include various input/output circuitry arranged in between the communication port 332 and the switch 320. When the ablation probe 104 is properly mounted to the holster device 300, the communication port 222 of the ablation probe 104 may align with and abut/contact the communication port 332 of the holster device 300, so as to establish communication between the holster device 300 and the upstream components of the system 100 (FIG. 1), such as the controller 114 (FIG. 1), via the ablation probe 104. In embodiments, the proximal portion 328 of the ablation probe 104 may be rotated into the opening 326 defined in the retainer 324, such that mounting the ablation probe 104 may include rotating the ablation probe 104 into the retainer 324 to thereby secure the ablation probe 104 to the holster device 300.

As with the communication port 222, the communication port 332 includes one or more electrical contacts or traces 334 (two shown) that are in communication with the switch 320 via various output circuitry and electronic components contained in the holster device 300. In the illustrated embodiment, the communication port 332 also includes one or more magnets 336 (two shown). The electrical contacts 334 are arranged in a single file row and the magnets 336 are arranged on opposite ends of the row of electrical contacts 334. The electrical contacts 334 and/or the magnets 336 may, however, be differently arranged, for example, to correspond and mate with the electrical contacts 224 and the magnets 226 of the communication port 222 on the ablation probe 104.

When the ablation probe 104 is properly mounted to the holster device 300, the magnets 336 align and mate (i.e., electrically attract each other) with the magnets 226 of the ablation probe 104, and may thus be operable to help seat the ablation probe 104 within the holster device 300 and maintain contact between the electrical contacts 224 of the ablation probe 104 and associated electrical contacts 334 contained within the holster device 300. In the illustrated embodiment, the electrical contacts 334 and the magnets 336 of the communication port 332 are arranged in the docking bay 306 of the cradle portion 304 such that they contact those of the communication port 222 when the ablation probe 104 is mounted within the docking bay 306.

When the ablation probe 104 is seated in the holster device 300, the communication port 222 is configured to locate, align, and mate with the communication port 332 of the holster device 300. Stated differently, when the ablation probe 104 is mounted to the holster device 300, the electrical contacts 234 of the ablation probe 104 align and mate (e.g., place in electrical or optical communication) with and contact the electrical contacts 334 of the holster device 300. In the illustrated embodiment, the electrical contacts 334 includes two electrical contacts/traces, with a first of the electrical contacts 334 connected to a first end of a circuit contained within the holster device 300, and with a second of the electrical contacts 334 connected to a second end of a circuit contained within the holster device. In operation, manual actuation of the switch 320 causes the circuit to open or close.

When the ablation probe 104 is initially seated in the docking bay 306 and connected to the holster device 300, the controller 114 may be configured to determine that the circuitry within the ablation probe 104 is in the “high state” (or the deactivated mode), and the controller 114 will cause the ablation probe 104 to be (or remain) in the “high state” (or the deactivated mode) until the user engages the switch 320 and moves the switch 320 into the activated position to thereby switch the ablation probe 104 into the “low state” (or activated mode). Thus, upon such activation of the switch 320, the controller 114 may be configured to then cause activation of the ablation probe 104.

In embodiments, the electrical contacts 334 may include one or more additional contacts. For example, in embodiments where the communication port 222 of the ablation probe 104 includes three electrical contacts 224, the communication port 332 may similarly include three electrical contacts 334 arranged to mate/contact the three electrical contacts 224 of the communication port 222. As detailed above, in an example where the electrical contacts 334 includes three electrical contacts 334, a third of the three electrical contacts 334 may be utilized to control additional functionality of the ablation probe 104, such as the switch 320 is operable to control flow of coolant flow to thereby activate the stick region 214 of the ablation probe 104 seated in the holster device 300.

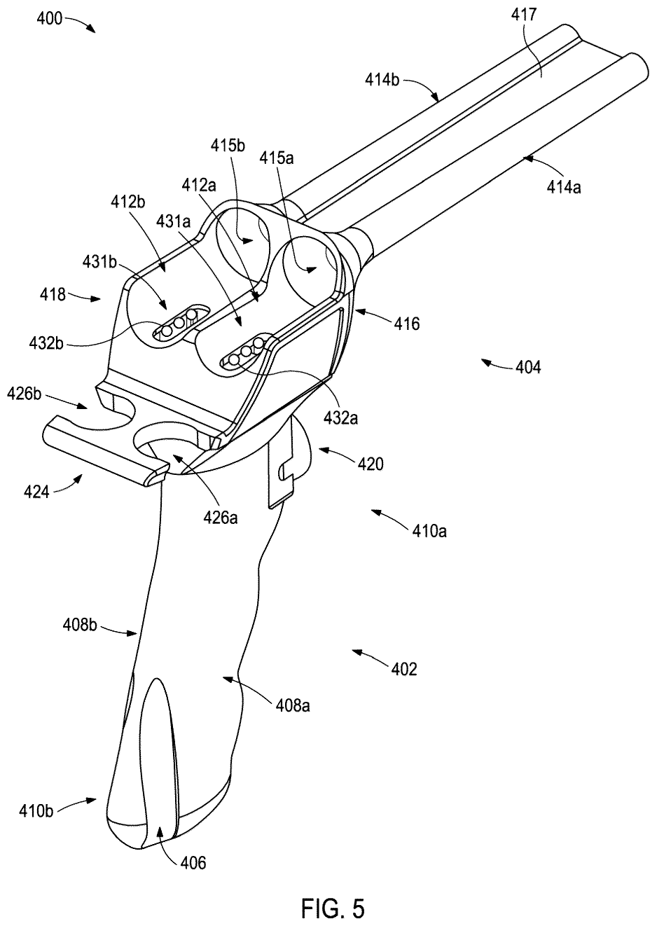

FIG. 5 depicts another example holster device 400, according to one or more alternate embodiments. In the illustrated embodiment, the holster device 400 is configured to receive and support a pair of ablation probes, and each ablation probe may be the same as or similar to the ablation probe 104 of FIG. 3. Thus, each ablation probe may include the communication port 222 (FIGS. 2-3) and the antenna 208 (FIGS. 1-3), as detailed above. The holster device 400 is configured to removably retain the ablation probes, side by side, such that the antennas of the ablation probes extend parallel to each other.

In the illustrated embodiment, the holster device 400 includes a handle portion 402 and a cradle portion 404. The handle portion 402 is configured to be held (grasped) by the user, and may thus employ an ergonomic geometry suitable for being received within the palm or hand of the user. In embodiments, the handle portion 402 includes or otherwise defines a slot 406 formed therein for receiving and retaining a cable/cord, such as the cable assembly 206 (FIGS. 2-3) and/or the cooling tube 212 (FIGS. 2-3), and thereby facilitate cable management and ensure cables/cords do not impede operation of the holster device 400 during a procedure.

In the illustrated embodiment, the handle portion 402 includes a first side 408a and a second side 408b opposite the first side 408a, and the slot 406 is a first slot formed in the first side 408a of the handle portion 402. The handle portion 402 may further includes or define a second slot (not shown) formed in the second side 408b of the handle portion 402. In this manner, the first slot 406 manages the cables/cords associated with one ablation probe and the second slot manages the cables/cords associated with the second ablation probe.

As shown, the handle portion 402 includes an upper end 410a and a lower end 410b opposite the upper end 410a, and the cradle portion 404 is attached to the handle portion 402 at the upper end 410a. The cradle portion 404 provides and otherwise defines a pair of docking bays 412a, 412b and a pair of cannulas 414a, 414b. As shown, the docking bays 412a, 412b each include a distal end 416 and a proximal end 418, and the cannulas 414a, 414b each extend distally from the distal end 416 of the corresponding docking bays 412a, 412b. More specifically, the first cannula 414a is structurally connected to and extends from the distal end 416 of the first docking bay 412a, and the second cannula 414b is structurally connected to and extends from the distal end 416 of the second docking bay 412b.

Each cannula 414a, 414b defines an inner lumen/bore 415a, 415b through which the probe cannula (e.g., the probe cannula 204) and the antenna (e.g., the antenna 208) of the corresponding ablation probe (e.g., the ablation probe 104) can extends when the ablation probe is connected (mounted) to the holster device 400. In some embodiments, as illustrated, a web 417 extends between the cannulas 414a, 414b to thereby provide support for the cannulas 414a, 414b and inhibit relative movement between them. In other embodiments, however, the web 417 may be omitted, without departing from the scope of the disclosure.

The holster device 400 further includes a switch 420 and a pair of communication ports 431a, 431b that are each operably connected to (in communication with) the switch 420. The communication ports 431a, 431b may be the same as or similar to the communication port 332 (FIG. 4), and may thus each include electrical contacts 432a, 432b, respectively. The electrical contacts 432a, 432b are in electrical communication with the switch 420.

In some embodiments, the holster device 400 may also include a retainer 424 to help inhibit accidental removal of the ablation probes from the holster device 400. As shown, the retainer 424 extends proximally from the proximal end 418 of the cradle portion 404 and includes or defines a pair of openings 426a, 426b within which the proximal portions 328 (FIG. 3) of a pair of side by side ablation probes may be retained. When the proximal portion of the ablation probe is received within either of the openings 426a, 426b, the retainer 424 at least partially surrounds the proximal portion to thereby inhibit removal of the ablation probe from the holster device 400 until sufficient force is applied to separate the proximal portion from the corresponding opening 426a, b and thereby overcome the snap fit configuration of the retainer 424. In embodiments, the proximal portion 328 of each ablation probe 104 utilized with the holster device 400 is rotated into one of the openings 426a, 426b defined in the retainer 424, such that mounting the ablation probes 104 may include rotating each ablation probe 104 into the retainer 424 to thereby secure the ablation probes 104 to the holster device 400.

When the proximal portion of an ablation probe is inserted into either of the openings 426a, 426b of the retainer 424, the proximal portion extends downward in a direction substantially similar (or parallel) to the handle portion 402 to thereby similarly direct the cable/cord (e.g., the cable assembly 206 and/or the cooling tube 212) downward in a direction substantially similar (or parallel) to the handle portion 302 (FIG. 4), and then the cable/cord may be inserted into the slot 406 to facilitate cable management. In particular, where a first ablation probe is mounted in the first docking bay 412a, the cable/cord associated therewith may be retained in the slot 406 on the first side 408a of the handle portion 402, and where a second ablation probe is mounted in the second docking bay 412b, the cable/cord associated with the second ablation probe may be retained in the second slot (not shown) formed in the second side 408b of the handle portion 402.

Also disclosed herein are methods of using and assembling the system 100. In embodiments, the method includes mounting an ablation probe (e.g., the ablation probe 104) on a holster device (e.g., the holster device 300 or 400), wherein the ablation probe includes an antenna (e.g., the antenna 208) and is in communication with a controller (e.g., the controller 114). The holster device is configured to removably receive the ablation probe, and the holster further comprises a switch (e.g., the switch 320 or 420). When the ablation probe is mounted in the holster device, the controller is operable to activate the ablation probe upon activation of the switch.

In embodiments, mounting the ablation probe on the holster device further comprises inserting the antenna through a lumen (e.g., the lumen 311, 415a, 415b). In embodiments, mounting the ablation probe on the holster device further comprises inserting the cable (e.g., the cable assembly 206 and/or the cooling tube 212) within a slot (e.g., the slot 406). In embodiments, mounting the ablation probe on the holster device further comprises inserting a proximal portion (e.g., the proximal portion 328) of the ablation probe within a retainer (e.g., the retainer 324, 424). In embodiments, the ablation probe is positioned within the docking bay defined in the holster device, and then the proximal portion (e.g., the proximal portion 328) of the ablation probe is rotated into the opening (e.g., the opening 326, 426a, 426b) defined in the retainer, such that mounting the ablation probe may include rotating the ablation probe into the retainer to thereby secure the ablation probe to the holster device. In embodiments, the ablation probe may be snapped into place within the holster device such that there is a tactile feeling felt by the user when the ablation probe is secured in place within the holster device. In embodiments, mounting the ablation probe on the holster device further comprises positioning the ablation probe such that the first electrical contact (e.g., electrical contacts 224) contacts the second electrical contact (e.g., electrical contacts 334, 432a, 432b). In embodiments, the force of the magnets arranged on the cradle portion of the holster device are attracted to the corresponding magnets arranged on the handle of the ablation probe to thereby pull the handle into position within the docking bay of the cradle portion and to thereby hold the orientation of the ablation probe relative to the holster device. Thus, in embodiments, mounting the ablation probe on the holster device may further comprise pulling the handle of the ablation probe into the cradle portion of the holster device via corresponding magnets and holding or maintaining orientation of the ablation probe relative to the holster device via such corresponding magnets.

Embodiments herein include:

A. A system, comprising: an ablation probe that includes an antenna; a controller in communication with the ablation probe; and a holster device configured to removably receive the ablation probe, the holster device including a switch, wherein, when the ablation probe is mounted to the holster device, the switch is placed in communication with the controller such that actuating the switch correspondingly activates the ablation probe.

B. A method, comprising: providing an ablation probe that includes: a handle housing; an antenna extending distally from the handle housing; and a first communication port mounted to the handle housing and including one or more first electrical contacts in communication with a controller; mounting the ablation probe to a holster device that includes: a docking bay sized to receive the handle housing; a switch; and a second communication port arranged within the docking bay and including one or more second electrical contacts in communication with the switch; aligning and mating the one or more first electrical contacts with the one or more second electrical contacts and thereby placing the switch in communication with the controller; and actuating the switch and thereby activating the ablation probe.

C. A system, comprising: an ablation probe that includes an antenna and a first electrical contact; a controller in communication with the first electrical contact; and a holster device having a switch and a second electrical contact in communication with the switch, wherein the holster is configured to removably receive the ablation probe and the second electrical contact is arranged to contact the first electrical contact when the ablation probe is mounted to the holster, and wherein the controller is operable to activate the ablation probe upon activation of the switch.

Each of embodiments A through C may have one or more of the following additional elements in any combination: Element 1: wherein the antenna outputs microwave energy upon actuating the switch and thereby activating the ablation probe. Element 2: wherein cooling fluid circulates through the ablation probe upon actuating the switch and thereby activating the ablation probe. Element 3: wherein the ablation probe includes one or more cables, and the holster device defines a slot configured to retain the one or more cables when the ablation probe is mounted to the holster device. Element 4: wherein the ablation probe comprises a first ablation probe and the system further includes a second ablation probe including a second antenna, and wherein the holster device is configured to removably receive the first and the second ablation probes such that the antenna of each ablation probe extends parallel to each other. Element 5: wherein the ablation probe includes a first communication port with one or more first electrical contacts in communication with the controller, and the holster device includes a second communication port with one or more second electrical contacts in communication with the switch, and wherein, when the ablation probe is mounted to the holster device, the one or more first electrical contacts align and mate with the one or more second electrical contacts. Element 6: wherein the first communication port includes one or more first magnets, and the second communication port includes one or more second magnets, and wherein, when the ablation probe is mounted to the holster device, the one or more first magnets are attracted to the one or more second magnets to releasably attach the ablation probe to the holster device. Element 7: wherein the switch comprises a spring loaded trigger that is movable between an activated position and an inactivated position, and the spring loaded trigger is biased to the inactivated position. Element 8: wherein the holster device further comprises a handle portion and a cradle portion, and wherein the cradle portion is configured to receive a handle housing of the ablation probe. Element 9: wherein the cradle portion includes a cannula through which the antenna of the ablation probe extends when the ablation probe is received within the cradle portion. Element 10: wherein the handle portion is configured to retain a cable of the ablation probe when the ablation probe is mounted on the holster device. Element 11: wherein the holster device includes a cannula that defines a lumen, and wherein mounting the ablation probe to the holster device comprises inserting the antenna through the lumen. Element 12: wherein the ablation probe further includes one or more cables, and the holster device further includes a handle portion that defines a slot, the method further comprising receiving and retaining the one or more cables in the slot when the ablation probe is mounted to the holster device. Element 13: wherein the holster device includes a cradle portion and a retainer extends from a proximal end of the cradle portion, and wherein mounting the ablation probe to the holster device further comprises inserting a proximal portion of the ablation probe within the retainer. Element 14: wherein the first communication port includes one or more first magnets, and the second communication port includes one or more second magnets, the method further comprising attracting the one or more first magnets to the one or more second magnets and thereby releasably attaching the ablation probe to the holster.

By way of non-limiting example, exemplary combinations applicable to A through C include: Element 5 with Element 6; Element 9 with Element 8; and Element 10 with Element 8.

Therefore, the disclosed systems and methods are well adapted to attain the ends and advantages mentioned as well as those that are inherent therein. The particular embodiments disclosed above are illustrative only, as the teachings of the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope of the present disclosure. The systems and methods illustratively disclosed herein may suitably be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The use of directional terms such as above, below, upper, lower, upward, downward, left, right, and the like are used in relation to the illustrative embodiments as they are depicted in the figures, the upward direction being toward the top of the corresponding figure and the downward direction being toward the bottom of the corresponding figure.

What is claimed is:

1. A system, comprising:
an ablation probe that includes an antenna;
a controller in communication with the ablation probe; and
a holster device configured to removably receive the ablation probe, the holster device including a switch,
wherein, when the ablation probe is mounted to the holster device, the switch is placed in communication with the controller such that actuating the switch correspondingly activates the ablation probe.

2. The system of claim 1, wherein the antenna outputs microwave energy upon actuating the switch and thereby activating the ablation probe.

3. The system of claim 1, wherein cooling fluid circulates through the ablation probe upon actuating the switch and thereby activating the ablation probe.

4. The system of claim 1, wherein the ablation probe includes one or more cables, and the holster device defines a slot configured to retain the one or more cables when the ablation probe is mounted to the holster device.

5. The system of claim 1, wherein the ablation probe comprises a first ablation probe and the system further includes a second ablation probe including a second antenna, and wherein the holster device is configured to removably receive the first and the second ablation probes such that the antenna of each ablation probe extends parallel to each other.

6. The system of claim 1, wherein the ablation probe includes a first communication port with one or more first electrical contacts in communication with the controller, and the holster device includes a second communication port with one or more second electrical contacts in communication with the switch, and wherein, when the ablation probe is mounted to the holster device, the one or more first electrical contacts align and mate with the one or more second electrical contacts.

7. The system of claim 6, wherein the first communication port includes one or more first magnets, and the second communication port includes one or more second magnets, and wherein, when the ablation probe is mounted to the holster device, the one or more first magnets are attracted to the one or more second magnets to releasably attach the ablation probe to the holster device.

8. The system of claim 1, wherein the switch comprises a spring loaded trigger that is movable between an activated position and an inactivated position, and the spring loaded trigger is biased to the inactivated position.

9. The system of claim 1, wherein the holster device further comprises a handle portion and a cradle portion, and wherein the cradle portion is configured to receive a handle housing of the ablation probe.

10. The system of claim 9, wherein the cradle portion includes a cannula through which the antenna of the ablation probe extends when the ablation probe is received within the cradle portion.

11. The system of claim 9, wherein the handle portion is configured to retain a cable of the ablation probe when the ablation probe is mounted on the holster device.

12. A method, comprising:
providing an ablation probe that includes:
a handle housing;
an antenna extending distally from the handle housing; and
a first communication port mounted to the handle housing and including one or more first electrical contacts in communication with a controller;
mounting the ablation probe to a holster device that includes:
a docking bay sized to receive the handle housing;
a switch; and
a second communication port arranged within the docking bay and including one or more second electrical contacts in communication with the switch;
aligning and mating the one or more first electrical contacts with the one or more second electrical contacts and thereby placing the switch in communication with the controller; and
actuating the switch and thereby activating the ablation probe.

13. The method of claim 12, wherein the holster device includes a cannula that defines a lumen, and wherein mounting the ablation probe to the holster device comprises inserting the antenna through the lumen.

14. The method of claim 12, wherein the ablation probe further includes one or more cables, and the holster device further includes a handle portion that defines a slot, the method further comprising receiving and retaining the one or more cables in the slot when the ablation probe is mounted to the holster device.

15. The method of claim 12, wherein the holster device includes a cradle portion and a retainer extends from a proximal end of the cradle portion, and wherein mounting the ablation probe to the holster device further comprises inserting a proximal portion of the ablation probe within the retainer.

16. The method of claim 12, wherein the first communication port includes one or more first magnets, and the second communication port includes one or more second magnets, the method further comprising attracting the one or more first magnets to the one or more second magnets and thereby releasably attaching the ablation probe to the holster.

17. A system, comprising:
an ablation probe that includes an antenna and a first electrical contact;
a controller in communication with the first electrical contact; and
a holster device having a switch and a second electrical contact in communication with the switch,
wherein the holster is configured to removably receive the ablation probe and the second electrical contact is arranged to contact the first electrical contact when the ablation probe is mounted to the holster, and
wherein the controller is operable to activate the ablation probe upon activation of the switch.

18. The system of claim 17, wherein the ablation probe comprises a first ablation probe and the system further includes a second ablation probe including a second antenna, and the holster device removably retains the first and the second ablation probes such that the antenna of each ablation probe extends parallel to each other.

19. The system of claim 17, wherein cooling fluid circulates through the ablation probe upon actuating the switch and thereby activating the ablation probe.

20. The system of claim 17, wherein the ablation probe includes one or more cables, and the holster device defines a slot configured to retain the one or more cables when the ablation probe is mounted to the holster device.
